(19) United States
(12) Patent Application Publication
(10) Pub. No.: US 2022/0265901 A1
(43) Pub. Date: Aug. 25, 2022

(54) METHOD FOR ENHANCING ACTIVITY IN A GRAFT

(71) Applicant: TERUMO KABUSHIKI KAISHA, Tokyo (JP)

(72) Inventors: Kenji OYAMA, Kanagawa (JP); Isamu MATSUDA, Kanagawa (JP)

(73) Assignee: TERUMO KABUSHIKI KAISHA, Tokyo (JP)

(21) Appl. No.: 17/681,877

(22) Filed: Feb. 28, 2022

Related U.S. Application Data
(63) Continuation of application No. PCT/JP2020/037063, filed on Sep. 30, 2020.

(30) Foreign Application Priority Data
Sep. 30, 2019 (JP) 2019-178999

(51) Int. Cl. A61L 27/38; C12N 5/00; A61L 27/58; C12M 1/12

(52) U.S. Cl. CPC A61L 27/3895; C12N 5/0068; A61L 27/58; C12M 25/14; C12N 2533/54

(57) ABSTRACT

A method for enhancing activity selected from the group consisting of cytokine production capacity, proliferation capacity, engraftment capacity, angiogenesis-inducing capacity, and tissue regeneration capacity in a graft, particularly in a sheet-shaped cell culture containing a somatic cell, involves incubating the graft at a temperature of 25° C. or higher.

### CROSS-REFERENCES TO RELATED APPLICATIONS

This application is a continuation of International Application No. PCT/JP2020/037063 filed on Sep. 30, 2020, which claims priority to Japanese Patent Application No. 2019-178999 filed on Sep. 30, 2019, the entire content of both of which is incorporated herein by reference.

### TECHNOLOGICAL FIELD

This disclosure relates to a method for enhancing activity in a graft, a method for production of a graft involving the method, a graft produced by the production method, and a method for treating a disease using the graft.

### BACKGROUND DISCUSSION

In recent years, attempts have been made to transplant various cells for the repair of damaged tissues and the like. For example, fetal cardiomyocytes, skeletal myoblast cells, mesenchymal stem cells, cardiac stem cells, ES cells, and iPS cells are used for experiments to repair myocardial tissues damaged by ischemic heart disease such as angina pectoris and myocardial infarction (Haraguchi et al., Stem Cells Transl Med. 2012 February; 1(2): 136-41).

As part of the experiments, a cell structure formed by utilizing a scaffold and a sheet-shaped cell culture having cells formed into a sheet have been developed (JP 2007-528755 A and Sawa et al., Surg Today. 2012 January; 42(2): 181-4). Such a sheet-shaped cell culture and other grafts have been studied for therapeutic applications. For example, cultured epidermal sheets are considered to be used for damaged skin caused by burns or the like, corneal epithelial sheet-shaped cell cultures are considered to be used for damaged corneas, and oral mucosa sheet-shaped cell cultures are considered to be used in endoscopic esophagectomy. Some of these grafts have been used clinically.

### SUMMARY

With the progress of clinical application of sheet-shaped cell cultures and other grafts, there has been demand for a graft with higher qualities that is easy to handle and easy to produce. It is known that various cytokines produced by cells in a graft act on a tissue of a host and stimulate regeneration of the tissue. However, in the study of methods to improve grafts, the present inventors have faced a problem that preservation of a graft diminishes activity such as cytokine production capacity and cell proliferation capacity in the graft.

The inventors have continued with the study to solve the problem and have found that incubating a sheet-shaped cell culture at 25° C. or higher enhances cytokine production capacity and activity in cells in the graft. In a further study, the inventors have found that a graft having a scaffold enhances cytokine production capacity while maintaining the shape of the graft.

The disclosure here relates to the following methods and graft.

<1> A method for enhancing activity selected from the group consisting of cytokine production capacity, proliferation capacity, engraftment capacity, angiogenesis-inducing capacity, and tissue regeneration capacity in a graft containing a somatic cell, the method involving incubating the graft at 25° C. or higher.

<2> The method according to <1>, in which the graft has a scaffold.

<3> The method according to <2>, in which the scaffold is a gel including fibrin, gelatin, or collagen.

<4> A method for production of a graft, the method involving the method according to any one of <1> to <3>.

<5> The method according to any one of <1> to <4>, in which the graft is a sheet-shaped cell culture.

<6> A graft obtained by the method according to any one of <1> to <3> or a graft produced by the method according to <4> or <5>.

<7> The graft according to <6> for treating a heart disease.

<8> A method for treating a disease to be ameliorated by application of a graft, the method involving applying the graft according to <6> or <7> to a subject in need thereof.

A graft as described herein has high activity such as cytokine production capacity and proliferation capacity and is excellent in engraftment to a tissue, in viability and functionality after the engraftment, and in persistence of the functions, which enables not only effective treatment of various diseases but also long-term preservation and transfer. Therefore, it is possible to offer a high-quality graft.

According to another aspect, a method for producing a graft comprises seeding cells on a substrate, producing a sheet-shaped cell culture using the seeded cells, the sheet-shaped cell culture forming a graft, applying a gel layer onto the graft, and incubating the graft at a temperature that is 25° C. or higher.

### BRIEF DESCRIPTION OF THE DRAWINGS

FIG. 1 shows the number of cells when a sheet-shaped cell culture containing skeletal myoblast cells formed with a fibrin gel layer is preserved in HBSS at 4° C., 25° C., 30° C., 35° C., and 37° C.; and

FIG. 2 shows the number of cells when a sheet-shaped cell culture containing skeletal myoblast cells formed with a fibrin gel layer is preserved in DMEM (10% of FBS) at 4° C., 25° C., 30° C., 35° C., and 37° C.

### DETAILED DESCRIPTION

Unless otherwise defined herein, all technical and scientific terms used herein have the same meaning as commonly understood by one of ordinary skill in the art. All patents, applications, published applications, and other publications referred to herein are hereby incorporated by reference in their entirety. In case of any inconsistency among or between the publications referred to herein and the description herein, the description herein shall govern.

<Method for Enhancing Activity in Graft>

This disclosure relates to a method for enhancing activity selected from the group consisting of cytokine production capacity, proliferation capacity, engraftment capacity, angiogenesis-inducing capacity, and tissue regeneration capacity in a graft, the method involving incubating the graft at 25° C. or higher.

The “graft” in this disclosure represents a structure to be transplanted into a living body, particularly, a structure for transplantation including cells as constituents. Cells included in the graft may be connected to each other directly (or via a cellular element such as adhesion molecule) and/or connected to each other via an intervening substance (hereinafter also referred to as “scaffold”). The cells are connected to each other at least physically (mechanically) but may be connected functionally, for example, chemically or electrically. Examples of the graft in this disclosure include, but are not limited to, a sheet-shaped cell culture, a cell agglomerate, a spheroid, and an organoid (herein, “cell agglomerate,” “spheroid,” and “organoid” are collectively referred to as “cell mass”). Preferably, the graft is a sheet-shaped cell culture or a spheroid, and more preferably a sheet-shaped cell culture.

The graft can include a scaffold. A scaffold includes cells attached to or embedded in a surface or an inner part thereof. Due to such a structure, scaffolds are used in the art to maintain physical integrity of a graft or to impart the graft with a strength. The scaffold is not particularly limited as long as it is a substance capable of connecting cells at least physically (mechanically) and may be a cell-derived substance or a substance other than cell-derived substances. Known examples of the scaffold include a biocompatible film or gel. Examples of the biocompatible film and gel include, but are not limited to, films such as amnion, PVDF, polytetrafluoroethylene (PTFE), polyurethane, polypropylene, polyester, vinyl chloride, polycarbonate, acrylic, silicone, MPC (2-methacryloyloxyethyl phosphorylcholine), polylactic acid, polyglycolic acid, and poly(lactic-co-glycolic acid) (PLGA) and gels containing fibrin, gelatin, sodium collagen alginate, temperature-responsive gel (trade name: Mebiol gel) obtained by crosslinking poly(N-isopropylacrylamide) (PNIPAAm) with polyethylene glycol (PEG), hyaluronic acid, glycosaminoglycan, proteoglycan, chondroitin, cellulose, agarose, carboxymethylcellulose, chitin, chitosan, atelocollagen, elastin, fibronectin, pronectin, laminin, tenascin, fibroin, entactin, thrombospondin, retrodine, dextrin, or trehalose. The scaffold is preferably a gel containing fibrin gel, gelatin, or collagen, and more preferably a gel containing fibrin gel.

Examples of the graft having a scaffold include, but are not limited to, a graft having a film on a surface or inner part thereof, a graft having a gel layer formed on a surface or inner part thereof, and a graft having integrity imparted by a gel filling a gap between a cell, cell mass, and sheet-shaped cell culture.

The “sheet-shaped cell culture” in this disclosure has cells connected to each other to form a sheet. The cells may be connected to each other directly (or via a cellular element such as adhesion molecule) and/or connected to each other via a scaffold.

Therefore, the “sheet-shaped cell culture” in this disclosure represents not only cultured cells having a sheet shape but also cells formed in a sheet shape. The sheet-shaped cell culture may include one cell layer (a single-layered cell culture) or may include two or more cell layers (a multiple-layered cell culture including two layers, three layers, four layers, five layers, or six layers). Furthermore, in the sheet-shaped cell culture, cells do not necessarily have a clear layer structure and may have a three-dimensional structure with a thickness exceeding a thickness of one cell. For example, in a perpendicular cross section of the sheet-shaped cell culture, cells may be arranged unevenly (for example, in a mosaic manner) without being aligned evenly in the horizontal direction or cells may include a scaffold such as film and gel.

The cells may be xenogeneic cells or syngeneic cells. When “xenogeneic cells” of a graft is used for transplantation, the “xenogeneic cells” represent cells derived from an organism of a species different from a recipient. For example, in a case where a human is a recipient, cells derived from a monkey or pig correspond to the xenogeneic cells. In contrast, “syngeneic cells” represent cells derived from an organism of the same species as a recipient. For example, in a case where a human is a recipient, human-derived cells correspond to the syngeneic cells. The syngeneic cells include autologous cells (also referred to as own cells or self cells) or recipient-derived cells, and allogeneic cells (also referred to as non-self cells). In this disclosure, it is preferable to use the autologous cells because transplanted autologous cells do not cause rejection. However, it is possible to use the xenogeneic cells or allogeneic cells. Using the xenogeneic cells or allogeneic cells may require immunosuppressive treatment to suppress rejection. Herein, cells other than the autologous cells, that is, the xenogeneic cells and allogeneic cells may be collectively referred to as non-autologous cells. In an embodiment of this disclosure, the cells are self cells or non-self cells. In an embodiment of this disclosure, the cells are self cells (including self iPS cells). In another embodiment of this disclosure, the cells are non-self cells (including non-self iPS cells).

Cells included in the graft of this disclosure are not particularly limited as long as the cells form a graft, particularly, a sheet-shaped cell culture, and the cells include, for example, somatic cells. In a preferred embodiment, the cells included in the graft are non-transgenic somatic cells. The cells included in the graft may be non-transgenic somatic cells. The cells included in the graft include adhesion cells (adherent cells). Examples of the adhesion cells include somatic cells (such as cardiomyocytes, fibroblast cells, epithelial cells, endothelial cells, hepatic cells, pancreatic cells, renal cells, adrenal cells, periodontal ligament cells, gingival cells, periosteal cells, skin cells, synovial cells, and cartilage cells) and stem cells (for example, tissue stem cells such as myoblast cells and cardiac stem cells; pluripotent stem cells such as embryonic stem cells and induced pluripotent stem (iPS) cells; and mesenchymal stem cells). The somatic cells may be differentiated from stem cells. Examples of the cells included in the graft include, but are not limited to, myoblast cells (such as skeletal myoblast cells), myosatellite cells, mesenchymal stem cells (such as cells derived from bone marrow, adipose tissue, peripheral blood, skin, hair root, muscular tissue, endometrium, placenta, and cord blood), cardiomyocytes, fibroblast cells, cardiac stem cells, synovial cells, cartilage cells, epithelial cells (such as oral mucosal epithelial cells, retinal pigment epithelial cells, and nasal mucosal epithelial cells), endothelial cells (such as vascular endothelial cells), hepatic cells (such as hepatic parenchymal cells), pancreatic cells (such as islet cells), renal cells, adrenal cells, periodontal ligament cells, gingival cells, periosteal cells, and skin cells.

The “myoblast cells” in this disclosure are progenitor cells of striated muscle cells and represent skeletal myoblast cells and cardiac myoblast cells (cardiac progenitor cells). The “skeletal myoblast cells” in this disclosure represent myoblast cells in a skeletal muscle. Skeletal myoblast cells are well known in the art and may be prepared from a skeletal muscle by any known method (for example, a method disclosed in JP 2007-89442 A) or may be commercially available (for example, Lonza, Cat #CC-2580). Skeletal myoblast cells are identified, for example, by markers such as CD56, α7 integrin, myosin heavy chain IIa, myosin heavy chain IIb, myosin heavy chain IId (IIx), MyoD, Myf5, Myf6, myogenin, desmin, and PAX3, but the markers are not limited thereto.

The “myosatellite cells (muscle satellite cells)” in this disclosure are progenitor cells of skeletal myoblast cells and are identified, for example, by markers such as CD56, CD34, Myogenin, Myf5, and Pax7, but the markers are not limited thereto. In a specific embodiment, the skeletal myoblast cells are CD56 positive. Skeletal myoblast cells and/or myosatellite cells may be derived from any organism having skeletal muscles. Examples of the organism include, but are not limited to, mammals such as humans, non-human primates, rodents (such as mice, rats, hamsters, and guinea pigs), rabbits, dogs, cats, pigs, horses, cows, goats, and sheep. In an embodiment, the skeletal myoblast cells and/or myosatellite cells are mammalian skeletal myoblast cells and/or myosatellite cells. In a specific embodiment, the skeletal myoblast cells and/or myosatellite cells are human skeletal myoblast cells and/or myosatellite cells.

The cells are preferably hepatic cells, fibroblast cells, myoblast cells, pancreatic cells, renal cells, vascular endothelial cells, or corneal epithelial cells, more preferably myoblast cells, and most preferably skeletal myoblast cells.

The cells included in the graft may be derived from any organism treatable with a graft. Examples of the organism include, but are not limited to, humans, non-human primates, dogs, cats, pigs, horses, goats, sheep, rodents (such as mice, rats, hamsters, and guinea pigs), and rabbits. In addition, the number of types of cells included in the sheet-shaped cell culture is not particularly limited. The sheet-shaped cell culture may be composed of one type of cells or may include two or more types of cells. In a case where the number of cells that form a sheet-shaped cell culture is two or more, a ratio (purity) of the greatest number of cells is 50% or more, preferably 60% or more, more preferably 70% or more, and still more preferably 75% or more at the end of producing the sheet-shaped cell culture.

The cells may be xenogeneic cells or syngeneic cells. When “xenogeneic cells” of a graft is used for transplantation, the “xenogeneic cells” represent cells derived from an organism of a species different from a recipient. For example, in a case where a human is a recipient, cells derived from a monkey or pig correspond to the xenogeneic cells. In contrast, “syngeneic cells” represent cells derived from an organism of the same species as a recipient. For example, in a case where a human is a recipient, human-derived cells correspond to the syngeneic cells. The syngeneic cells include autologous cells (also referred to as own cells or self cells) or recipient-derived cells, and allogeneic cells (also referred to as non-self cells). In this disclosure, it is preferable to use the autologous cells because transplanted autologous cells do not cause rejection. However, it is possible to use the xenogeneic cells or allogeneic cells. Using the xenogeneic cells or allogeneic cells may require immunosuppressive treatment to suppress rejection. Herein, cells other than the autologous cells, that is, the xenogeneic cells and allogeneic cells may be collectively referred to as non-autologous cells. In an embodiment of this disclosure, the cells are self cells or non-self cells. In an embodiment of this disclosure, the cells are self cells. In another embodiment of this disclosure, the cells are non-self cells.

It is possible to use a graft produced by any known method. In a case where the graft is a sheet-shaped cell culture, it is possible to use a sheet-shaped cell culture typically produced by a method involving seeding cells on a substrate and sheet-forming the seeded cells, but the sheet-shaped cell culture is not limited thereto.

In an embodiment, the graft having a scaffold may be one produced by any known method.

In a case where the graft has a scaffold, for example, it is possible to employ a sheet-shaped cell culture provided with a gel layer and produced by a method further involving forming a gel layer after the sheet-forming of the seeded cells. The gel layer is formed on the sheet-shaped cell culture by a known method, for example, by spraying liquid thrombin and then allowing the sheet-shaped cell culture to stand for a certain period of time (see JP 6495603 B2), but the method is not limited thereto.

In a case where the graft has a scaffold, it is possible to employ a graft imparted with integrity by a gel and produced by a method involving: placing a cell, cell mass, and/or sheet-shaped cell culture on a substrate with an intercellular gap involved; and filling the intercellular gap with the gel for sheet-forming.

In another embodiment, in a case where the graft has a scaffold, it is possible to employ a graft imparted with integrity by a gel and produced by a method involving precipitating a cell, cell mass, and/or sheet-shaped cell culture on a substrate; and sheet-forming the cell, cell mass, and/or sheet-shaped cell culture by the gel.

The sheet-forming by filling the gap with the gel or the sheet-forming by the gel is performed, for example, by gently adding the gel to the substrate having a surface provided with cells. The gel to be added is not limited in amount but is preferably added so that the graft has a thickness of, for example, 10 μm to 2000 μm, preferably 10 μm to 500 μm, more preferably 50 μm to 200 μm.

In an embodiment, the gel is generated by mixing two liquids, and any known method can be used for gelling with such a gel. Examples of the gelling include, but are not limited to, a method for simultaneously adding liquid fibrinogen and liquid thrombin, and a method for forming a fibrin gel by dripping liquid fibrinogen on cells and then spraying liquid thrombin (JP 6495603 B2).

According to an embodiment, when a gel is used as a scaffold, the gel is a biodegradable gel and is decomposed in vivo, absorbed into the body, metabolized, and excreted. Examples of the gel include a fibrin gel obtained by mixing liquid fibrinogen and liquid thrombin, and an adhesion inhibitor obtained by mixing an aqueous solution of NHS-modified CM dextrin and trehalose with an aqueous solution of sodium carbonate and sodium hydrogen carbonate. These examples are commercially available gels as Bolheal (registered trademark) for tissue adhesion (available from Teijin Pharma Limited), Beriplast (registered trademark) for tissue adhesion (available from CSL Behring), and AdSpray (registered trademark) (available from Terumo Corporation).

This disclosure involves incubating the graft at 25° C. or higher. The incubating is not particularly limited in time unless activity in a sheet-shaped cell culture is diminished and is about 1 hour to about 96 hours, preferably about 4 hours to about 72 hours, more preferably about 5 hours to about 72 hours, and still more preferably about 8 hours to about 48 hours.

Furthermore, the incubating is not particularly limited in temperature unless activity in the graft is diminished. For example, the incubation temperature is about 20° C. to about 45° C. The lower limit of the incubation temperature is, for example, about 20° C. or more, 21° C. or more, 22° C. or more, 23° C. or more, 24° C. or more, 25° C. or more, about 26° C. or more, about 27° C. or more, about 28° C. or more, about 29° C. or more, about 30° C. or more, about 31° C. or more, about 32° C. or more, about 33° C. or more, about 34° C. or more, about 35° C. or more, about 36° C. or more, about 37° C. or more, about 38° C. or more, about 39° C. or more, about 40° C. or more, about 41° C. or more, about 42° C. or more, about 43° C. or more, about 44° C. or more, or about 45° C. or more.

The upper limit of the incubation temperature is, for example, about 26° C. or less, about 27° C. or less, about 28° C. or less, about 29° C. or less, about 30° C. or less, about 31° C. or less, about 32° C. or less, about 33° C. or less, about 34° C. or less, about 35° C. or less, about 36° C. or less, about 37° C. or less, about 38° C. or less, about 39° C. or less, about 40° C. or less, about 41° C. or less, about 42° C. or less, about 43° C. or less, about 44° C. or less, about 45° C. or less, preferably 39° C. or less. The incubation temperature may be set by any combination of the above examples of the upper and lower limits.

In other words, the incubation is performed at a temperature of, for example, about 20° C. to about 45° C., about 25° C. to about 45° C., about 26° C. to about 44° C., from 27° C. to about 43° C., from 28° C. to 42° C., about 29° C. to about 41° C., about 30° C. to about 40° C., about 31° C. to about 39° C., about 32° C. to about 38° C., about 33° C. to about 37° C., about 34° C. to about 36° C. Preferably, the incubation is performed at a temperature of 27° C. to about 43° C., more preferably about 30° C. to about 40° C., still more preferably about 32° C. to about 39° C., still more preferably about 33° C. to about 39° C., and particularly preferably about 34° C. to about 39° C.

Incubating at 25° C. or higher may be performed at any timing after the production of the graft. For example, the incubating may be performed immediately after the production of the graft. Alternatively, for example, the incubating may be performed after the graft is preserved at 4° C. and then transferred to a different facility or may be performed in the facility after the graft is transferred thereto. However, it is preferable to incubate continuously immediately after the production.

According to an embodiment, in a case where the forming of a gel layer is added prior to the incubating, the gel layer is preferably formed prior to, for example, preserving of the graft at 4° C.

The graft may remain adhered to a substrate (for example, a culture substrate). Alternatively, the graft may be detached from the substrate. In a case where the graft remains adhered to the substrate, the incubating is performed after adding any medium to the substrate (or adding to a vessel when the substrate is a vessel surface). In a case where the graft is detached from the substrate, the incubating is performed after adding a medium to the vessel containing the graft.

The medium is not particularly limited as long as the medium ensures survival of the cells included in the graft. For example, saline, various physiological buffers (such as PBS and HBSS), and those based on various basal media for cell culture may be used. Examples of the basal medium include, but are not limited to, DMEM, MEM, F12, DME, RPMI1640, MCDB (such as MCDB 102, 104, 107, 120, 131, 153, and 199), L15, SkBM, RITC 80-7, and DMEM/F12. Many of these basal media are commercially available, and compositions of these basal media are known. The basal medium may be used with a standard composition (for example, in a commercially available state) or may be appropriately changed in composition according to types and conditions of cells. The medium may contain additives such as normal serums (for example, bovine serum such as fetal bovine serum (FBS), horse serum, and human serum) and various growth factors (such as FGF, EGF, VEGF, and HGF). A preferred example of the medium is DMEM, and particularly preferred example is DMEM containing 10% FBS.

The method may be performed in order to maintain or enhance activity in a graft such as cytokine production capacity (cytokine production capability) and proliferation capacity (proliferation capability) or may be performed in order to reactivate, maintain, or enhance activity in a sheet-shaped cell culture or the like whose activity is diminished by preservation or transfer. Furthermore, the method may be performed in order to reactivate, maintain, or enhance activity after repairing a graft that is partially broken during the production of the graft or, for example, after forming a gel layer.

The graft used in a method of the present disclosure has higher activity than a graft not subjected to the incubation step (which may hereinafter be referred to as “control graft”). Examples of the activity include, but are not limited to, cytokine production capacity, proliferation capacity, engraftment capacity, angiogenesis-inducing capacity, and tissue regeneration capacity. Herein, the “higher activity” indicates that the capacity is, for example, but is not limited to, 5% or more, 10% or more, 20% or more, 30% or more, 40% or more, 50% or more, 60% or more, 70% or more, 80% or more, 90% or more, 100% or more, 200% or more, 300% or more, or 400% or more based on the activity of the control graft.

According to an embodiment, cytokines are beneficial for engraftment of a graft into a tissue of a host. According to an embodiment, cytokines are beneficial for angiogenesis. According to an embodiment, cytokines are beneficial for induction of bone marrow mesenchymal stem cells. According to an embodiment, cytokines are beneficial for regeneration of tissues. Cytokines are known in the art, which enables one skilled in the art to determine an appropriate cytokine based on known information. According to an embodiment, a cytokine contains a growth factor. The cytokine can be selected from the group consisting of VEGF, HGF, SDF-1, FGF, and SCF. Therefore, the cytokine production capacity according to an embodiment is a capacity to produce a growth factor. Furthermore, the cytokine production capacity according to an embodiment is a capacity to produce a growth factor selected from the group consisting of VEGF, HGF, SDF-1, FGF, and SCF.

Activity in a graft is quantified by various techniques. The cytokine production capacity, for example, is quantified by culturing a graft obtained by a method of the present disclosure in a predetermined culture solution for a predetermined time and by measuring an amount of a cytokine secreted into the culture solution or measuring an expression level of cytokine genes in the graft. A technique known in the art can be used for measuring an amount of a specific protein or a gene expression level.

The proliferation capacity, for example, is quantified by measuring the number of viable cells in a graft included in the graft obtained by a method of the present disclosure or by measuring the number of surviving cells in a graft preserved in a culture solution for a predetermined time. A technique known in the art can be used for measuring the number of viable cells in the graft.

The engraftment capacity, for example, is quantified by applying a graft or a sheet-shaped cell culture to a tissue, observing conditions of the graft after a predetermined time, observing adhesion of the graft to the tissue and size, color, form of remaining graft, and by scoring these conditions. The angiogenesis-inducing capacity, for example, is quantified by applying a graft to a tissue, observing conditions of the graft and the tissue (such as the presence or absence of angiogenesis) at an application site after a predetermined time, and by scoring these conditions. Furthermore, the tissue regeneration capacity, for example, is quantified by applying a graft to a tissue, observing conditions of the tissue (such as the size of the tissue, the microstructure of the tissue, a ratio between damaged and normal parts of the tissue, a degree of induction of cardiomyocytes in bone marrow mesenchymal stem cells, and the function of the tissue) at an application site (host) after a predetermined time, and then, by scoring these conditions.

According to an embodiment, the graft having a scaffold is less likely to twist and/or shrink than a graft having no scaffold but subjected to incubating under similar conditions to the incubation disclosed here.

In this disclosure, “twist” and “shrink” of a graft indicate that an outer edge of the graft (for example, a sheet-shaped cell culture) is twisted or that the graft is curled. The “twist” and “shrink” represent wrinkles and folds physically caused by not only an action of intercellular adhesion but also by transfer. The extent of “twist” and “shrink” may be quantified, for example, by observing the shape, size, and appearance of the graft by a known method and by scoring these conditions.

All steps of the method according to an embodiment are performed in vitro. In another embodiment, the method involves, but is not limited to, a step performed in vivo, for example, harvesting cells or a tissue serving as a source of cells from a subject. All steps of the method according to an embodiment are performed under aseptic conditions.

<Method for Production of Graft Having High Activity>

Another aspect of the present disclosure relates to a method for production of a graft involving a method described herein.

The method for production of a graft according to an embodiment of the present disclosure involves, for example, but is not limited to, seeding cells on a substrate, sheet-forming the seeded cells, and incubating the cells at 25° C. or higher.

Furthermore, after the sheet-forming of the seeded cells, the sheeted graft may be detached, and then, incubated. Alternatively, the sheeted graft may be incubated while adhering to the culture substrate. Still further, after the sheet-forming of the seeded cells, a gel layer may be formed on the graft. In this case, the gel layer may be formed after detaching the graft or while the graft adheres to the substrate.

The method for production of a graft according to an embodiment of the present disclosure involves, but is not limited to, placing a cell, cell mass, and/or sheet-shaped cell culture on a substrate with an intercellular gap involved; filling the intercellular gap with a gel to form a sheet; and incubating at 25° C. or higher.

In another embodiment, the method for production of a graft involves, but is not limited to, precipitating a cell, cell mass, and/or sheet-shaped cell culture on a substrate; sheet-forming the cell, cell mass, and/or sheet-shaped cell culture by a gel; and incubating at 25° C. or higher.

In these embodiments, after filling the gap with the gel to form a sheet or after sheet-forming the sheet-shaped cell culture by the gel, the sheeted graft may be detached, and then, incubated. Alternatively, the sheeted graft may be incubated while adhering to the culture substrate.

Each of these steps can be carried out by any known technique suitable for the production of various grafts. The method may further involve producing a graft, and the producing of a graft may optionally involve one step or more.

The substrate, or a culture substrate, used for the production of a graft is not particularly limited as long as the substrate enables cells to form a graft thereon. Examples of the substrate include a vessel and a solid or semi-solid surface of the vessel. The vessel may include various kinds of materials and/or may have various shapes. The vessel preferably has a structure or material that does not allow a culture solution or other liquids to permeate therethrough. Examples of the material include, but are not limited to, polyethylene, polypropylene, Teflon (registered trademark), polyethylene terephthalate, polymethyl methacrylate, nylon 6,6, polyvinyl alcohol, cellulose, silicon, polystyrene, glass, polyacrylamide, polydimethylacrylamide, and metals (such as iron, stainless steel, aluminum, copper, and brass). Furthermore, the vessel preferably includes at least one flat surface. An example of the vessel includes, but is not limited to, a culture vessel provided with a bottom face including a substrate that allows formation of a cell culture and with a liquid-impermeable side face. Specific examples of the culture vessel include, but are not limited to, cell culture dishes and cell culture bottles. The bottom face of the vessel may be transparent or opaque. A vessel with a transparent bottom face makes it possible to observe cells or count the number of cells from the bottom of the vessel. The vessel may also have a solid or semi-solid surface inside. Examples of the solid surface include plates and vessels including various kinds of materials as described above. Examples of the semi-solid surface include gels and soft polymer matrices. The substrate may be prepared using a material from the above examples or a commercially available material.

Preferred examples of the substrate include, but are not limited to, those having an adhesive surface, a low adhesive surface, and/or a uniform well structure which are suitable for the formation of a graft. Specifically, in forming a sheet-shaped cell culture, examples of the substrate include those having a surface coated with a hydrophilic compound such as polystyrene treated by corona discharge, collagen gel, and a hydrophilic polymer, those having a surface coated with an extracellular matrix such as collagen, fibronectin, laminin, vitronectin, proteoglycan, and glycosaminoglycan, and those having a surface coated with a cell adhesion molecule such as cadherin family, selectin family, and integrin family. These examples of the substrate are commercially available (for example, Corning (registered trademark) TC-Treated Culture Dish, Corning). Furthermore, in forming a spheroid, examples of the substrate include those having a surface coated with a non-cell-adhesive compound or hydrogel such as soft agar, a temperature-responsive gel (trade name: Mebiol Gel) obtained by crosslinking poly(N-isopropylacrylamide) (PNIPAAm) with polyethylene glycol (PEG), polyhydroxyethyl methacrylate (polyHEMA), and a 2-methacryloyloxyethyl phosphoruscorine (MPC) polymer, and/or those having a surface with a structure including recesses and protrusions in a regular pattern. These examples of the substrate are also commercially available (for example, EZSPHERE (registered trademark)). The substrate may be transparent or opaque in whole or part.

The surface of the substrate may be coated with a material having physical properties that are changed according to temperature, light, and other stimuli. Examples of the material include, but are not limited to, known materials such as temperature-responsive materials including a homopolymer or copolymer of a (meth)acrylamide compound, an N-alkyl-substituted (meth)acrylamide derivative (such as N-ethylacrylamide, N-n-propylacrylamide, N-n-propylmethacrylamide, N-isopropylacrylamide, N-isopropylmethacrylamide, N-cyclopropylacrylamide, N-cyclopropylmethacrylamide, N-ethoxyethylacrylamide, N-ethoxyethylmethacrylamide, N-tetrahydrofurfurylacrylamide, and N-tetrahydrofurfurylmethacrylamide), an N,N-dialkyl-substituted (meth)acrylamide derivative (such as N,N-dimethyl (meth)acrylamide, N,N-ethylmethylacrylamide, and N,N-diethylacrylamide), a (meth)acrylamide derivative having a cyclic group (such as 1-(1-oxo-2-propenyl)-pyrrolidine, 1-(1-oxo-2-propenyl)-piperidine, 4-(1-oxo-2-propenyl)-morpholine, 1-(1-oxo-2-methyl-2-propenyl)-pyrrolidine, 1-(1-oxo-2-methyl-2-propenyl)-piperidine, 4-(1-oxo-2-methyl-2-propenyl)-morpholine), or a vinyl ether derivative (such as methyl vinyl ether) and photoresponsive materials such as a light-absorbing polymer having an azobenzene group, a copolymer of a vinyl derivative of triphenylmethane leukohydroxide and an acrylamide monomer, and an N-isopropylacrylamide gel containing spirobenzopyran (see, for example, JP 2-211865 A and JP 2003-33177 A). Application of a predetermined stimulus to these materials changes their physical properties such as hydrophilicity and hydrophobicity, thereby promoting detachment of a cell culture adhering to the materials. Culture dishes coated with temperature-responsive materials are commercially available (for example, UpCell (registered trademark) of CellSeed Inc. or Cepallet (registered trademark) of DIC Corporation) and may be employed in an embodiment of the present disclosure.

The substrate may have a shape of various kinds. The substrate is not particularly limited in area and may have an area of, for example, about 1 cm² to about 200 cm², about 2 cm² to about 100 cm², and about 3 cm² to about 50 cm². An example of the substrate includes a circular culture dish having a diameter of 10 cm. In this case, the substrate has an area of 56.7 cm². A culture surface may be flat or may have a structure including recesses and protrusions. In the latter case, it is preferable that the recesses and protrusions be in a regular pattern.

The substrate may be coated with a serum. A substrate coated with a serum makes it possible to form a denser graft, particularly, a denser sheet-shaped cell culture. The expression “coated with a serum” indicates that serum components adhere to a surface of the substrate. Such a condition can be obtained by, for example, treating the substrate with a serum, but the treating is not limited to the technique. The treating with a serum involves contacting the serum with the substrate and optionally incubating for a predetermined period of time.

As the serum, xenogeneic serums and/or syngeneic serums may be used. When a graft, specifically, a sheet-shaped cell culture is used for transplantation, the “xenogeneic serums” represent serums derived from organisms of a species different from a recipient. For example, in a case where a human is a recipient, serums derived from bovine or horses such as fetal bovine serum (FBS, FCS), calf serum (CS), and horse serum (HS) correspond to the xenogeneic serums. In contrast, “syngeneic serums” represent serums derived from organisms of the same species as a recipient. For example, in a case where a human is a recipient, human-derived serums correspond to the syngeneic serums. The syngeneic serums include autologous serums (also referred to as self serums), that is, serums derived from the recipient and allogeneic serums derived from allogeneic individuals other than the recipient. Herein, serums other than the autologous serums, that is, the xenogeneic serums and allogeneic serums may be collectively referred to as non-autologous serums.

The serum for coating the substrate is commercially available or prepared by an ordinary method from blood sampled from a desired organism. Specifically, for example, a blood sample is allowed to stand at room temperature for about 20 minutes to about 60 minutes to be coagulated, and the coagulated blood is centrifuged at about 1000×g to about 1200×g, thereby sampling supernatant.

When incubating on the substrate, the serum may be used in undiluted form or diluted form. The dilution is performed with any medium such as water, saline, various buffers (for example, PBS and HBSS), and various liquid media (for example, DMEM, MEM, F12, DMEM/F12, DME, RPMI 1640, MCDB (MCDB 102, 104, 107, 120, 131, 153, 199), L15, SkBM, and RITC 80-7), but the medium is not limited thereto. A dilute concentration is not particularly limited as long as serum components adhere to the substrate and is, for example, about 0.5% to about 100% (v/v), preferably about 1% to about 60% (v/v), and more preferably about 5% to about 40% (v/v).

The incubation is not particularly limited in time as long as the serum components adhere to the substrate. For example, the incubation lasts from about 1 hour to about 72 hours, preferably from about 2 hours to about 48 hours, more preferably from about 2 hours to about 24 hours, and still more preferably from about 2 hours to about 12 hours. The incubation is not particularly limited in temperature as long as the serum components adhere to the substrate. For example, the incubation is performed at a temperature of about 0° C. to about 60° C., preferably about 4° C. to about 45° C., and more preferably room temperature to about 40° C.

The serum may be discarded after the incubation. The serum is discarded by a commonly used technique for discarding a liquid such as suction with a pipette and decantation. In a preferred embodiment of this disclosure, after discarding the serum, the substrate may be washed with a serum-free washing solution. The serum-free washing solution is not particularly limited as long as it is a liquid medium that does not badly affect serum components adhering to the substrate. Examples of the washing solution include, but are not limited to, water, saline, various buffers (for example, PBS and HBSS), various liquid media (for example, DMEM, MEM, F12, DMEM/F12, DME, RPMI 1640, MCDB (MCDB 102, 104, 107, 120, 131, 153, 199), L15, SkBM, and RITC 80-7). The washing may employ a commonly used technique. For example, a serum-free cleaning solution is added to the substrate, the solution is stirred for a predetermined time (for example, about 5 seconds to about 60 seconds), and then, the solution is discarded. However, the washing is not limited to the technique.

The substrate in this disclosure may be coated with a growth factor. Herein, the “growth factor” represents any substance that stimulates proliferation of cells as compared with a substrate with no growth factor. Examples of the growth factor include epidermal growth factor (EGF), vascular endothelial growth factor (VEGF), and fibroblast growth factor (FGF). The techniques of coating, discarding, and washing of the substrate using a growth factor are basically similar to the techniques using a serum except that a dilute concentration during the incubation is, for example, about 0.0001 μg/mL to about 1 μg/mL, preferably about 0.0005 μg/mL to about 0.05 μg/mL, and more preferably about 0.001 μg/mL to about 0.01 μg/mL.

The substrate in this disclosure may be coated with a steroid. The “steroid” herein represents a compound having a steroid nucleus which may have negative effects on a living body such as adrenal insufficiency and Cushing's syndrome. Examples of the compound include, but are not limited to, cortisol, prednisolone, triamcinolone, dexamethasone, and betamethasone. The techniques of coating, discarding, and washing of the substrate using a steroid are basically similar to the techniques using a serum except that a dilute concentration of, for example, dexamethasone during the incubation is about 0.1 μg/mL to about 100 μg/m L, preferably about 0.4 μg/mL to about 40 μg/m L, and more preferably about 1 μg/mL to about 10 μg/mL.

The substrate may be coated with any one of a serum, a growth factor, and a steroid or any combination thereof, that is, a combination of serum and growth factor, a combination of serum and steroid, a combination of serum and growth factor and steroid, and a combination of growth factor and steroid. In a case where a plurality of components is used for coating, the components may be mixed and used for coating simultaneously or may be used for coating separately.

The culture solution used in a production method of the present disclosure is not particularly limited as long as the culture solution ensures survival of cells. Typically, the production method employs a culture solution containing an amino acid, a vitamin, or an electrolyte as a main component. The culture solution according to an embodiment is a basal medium-based culture solution for cell culture. Examples of the basal medium include, but are not limited to, DMEM, MEM, F12, DMEM/F12, DME, RPMI1640, MCDB (such as MCDB 102, 104, 107, 120, 131, 153, and 199), L15, SkBM, and RITC 80-7. Many of these basal media are commercially available, and compositions of these basal media are known. However, in the production methods of the present disclosure, compositions of the basal medium may be changed appropriately according to the type and conditions of cells.

Cells including graft-forming cells such as sheet-forming cells are seeded on the substrate. The graft-forming cells are not particularly limited as long as the cells are selected from the aforementioned examples that form a graft, particularly, a sheet-shaped cell culture. The cells include at least one graft-forming cell but may include two or more graft-forming cells or may include cells other than graft-forming cells. In another embodiment of this disclosure, at least one graft-forming cell included in a cell population is a skeletal myoblast cell. In these embodiments, the cells include myosatellite cells. That is, the cells include a sheet-shaped cell culture containing skeletal myoblast cells and myosatellite cells as sheet-forming cells. In these embodiments, the cells may include fibroblast cells. In another embodiment of this disclosure, at least one graft-forming cell included in a cell population is a mesenchymal stem cell. In these embodiments, the cells may also include vascular endothelial cells.

A density of cells to be seeded is not particularly limited as long as the density enables formation of a graft. In a preferred embodiment, a cell population is seeded at a density reaching confluence or higher. In this disclosure, the “density reaching confluence” refers to an inferable density at which seeded cells cover the entire adhesion surface of a culture vessel without any space. For example, when cells are seeded, the “density reaching confluence” refers to a density at which the cells are inferred to contact each other, a density at which contact inhibition occurs, or a density at which cell proliferation is substantially stopped by contact inhibition.

A seeding density of the cell population ranges from, but is not limited from, about 7.1×10⁵ cells/cm² to about 3.0×10⁶ cells/cm², about 7.3×10⁵ cells/cm² to about 2.8×10⁶ cells/cm², about 7.5×10⁵ cells/cm² to about 2.5×10⁶ cells/cm², about 7.5×10⁵ cells/cm² to about 3.0×10⁶ cells/cm², about 7.8×10⁵ cells/cm² to about 2.3×10⁶ cells/cm², about 8.0×10⁵ cells/cm² to about 2.0×10⁶ cells/cm², about 8.5×10⁵ cells/cm² to about 1.8×10⁶ cells/cm², or about 9.0×10⁵ cells/cm² to about 1.6×10⁶ cells/cm². Unless otherwise specified, note that these densities are applied to all cells contained in the cell population.

In another embodiment, seeding can be carried out in a culture solution substantially free of growth factors at a density at which at least one graft-forming cell included in the cell population does not substantially proliferate. In these embodiments, other cells included in the cell population may be seeded at a density that enables proliferation even though proliferation inhibition occurs. The substrate used in the method according to this disclosure is as already described earlier. In a preferred embodiment, the substrate may be coated with a serum. In another preferred embodiment, the substrate may be coated with a temperature-responsive material. In another preferred embodiment, the substrate may be coated with a temperature-responsive material and a serum.

<Kit>

Another aspect of the present disclosure relates to a kit for producing a graft, including some or all elements for production of a graft, particularly, for production of a graft without proliferation.

The kit may include, but is not limited to, for example, a material for forming a gel used in forming a gel layer, a solution containing fibrin and a solution containing thrombin as well as cells included in a graft (such as cryopreserved cells, cells collected by a method for collecting cells according to the present disclosure), a culture solution, a culture dish, instruments (such as pipette, dropper, and tweezer), and instructions on a method for producing a graft (for example, a manual or a medium such as flexible disk, CD, DVD, Blu-ray disk, memory card, and USB memory that stores information associated with the method for producing a graft and the method for collecting cryopreserved cells according to the present disclosure).

<Graft Having High Activity>

Another aspect of the present disclosure relates to a graft obtained by a method as described herein or a graft obtained by a method for production of a graft as described herein. According to an embodiment, a graft produced by the production method of this disclosure includes skeletal myoblast cells, myosatellite cells, fibroblast cells, mesenchymal stem cells, and/or parietal cells.

The graft obtained by the production methods of the present disclosure has higher activity than a control graft. The details of the expression “higher activity” are as already described earlier. Compared with the control graft, the graft according to an embodiment has high activity selected from the group consisting of cytokine production capacity, proliferation capacity, engraftment capacity, angiogenesis-inducing capacity, and tissue regeneration capacity. The graft according to an embodiment has a higher capacity than the control graft to produce a cytokine selected from the group consisting of HGF, SDF-1, FGF, SCF, and VEGF. Due to the high activity, the graft shows more advantageous effects than the control graft in various therapeutic applications. Particularly, a high capacity to produce HGF or VEGF stimulates engraftment to a tissue, angiogenesis induction, and tissue regeneration, and a high proliferation capacity enables a graft to function continuously over a long period of time, which enhances therapeutic effects. The graft according to an embodiment is obtained by the production methods described herein and has a scaffold which is less likely to twist and/or shrink compared with a graft with no scaffold. Therefore, compared with the control graft with no scaffold, the graft having a scaffold not only enhances continuous and long-term therapeutic effects such as engraftment to a tissue, angiogenesis induction, and tissue regeneration stimulatory action but also has a high strength, excellent operability and is easily applied to an affected area, which causes a small operational difference among practitioners having different levels of expertise. Accordingly, the graft having a scaffold reliably treats a disease and is much more convenient than the control graft with no scaffold.

<Method for Treating Disease>

Another aspect of this disclosure relates to a method for treating a disease in a subject, the method involving applying an effective dose of a graft obtained by the method of this disclosure or a method for production of a graft involving the method to the subject in need of the effective dose of the graft. Examples of the disease to be treated are as described above.

In this disclosure, the term “treatment” represents all types of medically acceptable prophylactic and/or therapeutic interventions aimed at curing, temporary remission, or prophylaxis of a disease. For example, the term “treatment” represents medically acceptable interventions for a variety of purposes including retardation or stoppage of the progression of a disease associated with tissue abnormalities, regression or elimination of a lesion, and prevention of the onset of the disease or prevention of disease recurrence.

In the treatment of this disclosure, the graft of this disclosure may be used together with, for example, components that enhance the viability, engraftment, and/or function of the graft and other active constituents useful for the treatment of a target disease.

The treatment of this disclosure may further involve producing a graft of this disclosure having activity enhanced by the method of this disclosure. Before producing a graft, the treatment of this disclosure may further involve harvesting cells (such as skeletal myoblast cells) or a tissue as a source of cells (such as skeletal myoblast cells) for producing a graft from a subject. In an embodiment, a subject from which cells or a tissue as a source of cells is harvested is the same individual as the subject receiving administration of a cell culture, composition, graft, or the like. In another embodiment, a subject from which cells or a tissue as a source of cells is harvested is a different individual of the same type as the subject receiving administration of a cell culture, composition, graft, or the like. In another embodiment, a subject from which cells or a tissue as a source of cells is harvested is an individual different from the subject receiving administration of a graft or the like.

The effective dose in this disclosure is, for example, a quantity that suppresses the onset or recurrence of a disease, alleviates symptoms, or retards or stops the progression of the disease (for example, the size, weight, and number of sheet-shaped cell cultures) and is preferably a quantity that prevents the onset and recurrence of a disease or cures the disease. In addition, it is preferable to apply a quantity that does not cause adverse effects exceeding the benefits of administration. The effective dose can be appropriately determined by, for example, a test in a laboratory animal such as mouse, rat, dog, and pig or a disease model animal. Such a test is well known to those skilled in the art. The size of a tissue lesion to be treated is also an important indicator for determining the effective dose.

Examples of the administration include intravenous administration, intramuscular administration, intraosseous administration, intrathecal administration, and direct application to a tissue. The administration is typically performed in a single dose. However, in a case where single-dose administration is not effective, the administration may be performed multiple times. When applying to a tissue, the cell culture, composition, graft, or the like of the present disclosure may be fixed to the tissue of a subject by a locking device such as sutures or staples.

### EXAMPLES

Aspects of the present disclosure will be described in more detail with reference to the following specific examples, which are not intended to limit the disclosure.

<Preparation of Skeletal Myoblast Cells>

Cells obtained from a skeletal muscle tissue aseptically harvested from a human adult thigh were seeded in a culture flask and proliferated in MCDB 131 containing 20% FBS. The proliferated cells were detached from the culture flask with a protease solution, collected, and then, concentrated by centrifugation, thereby obtaining cells containing skeletal myoblast cells. The obtained cells had a CD56 positive rate of about 85%.

### Comparative Example 1

### Production of Sheet-Shaped Cell Culture

A cell population containing skeletal myoblast cells cryopreserved in a cryopreservation solution (MCDB containing 10% DMSO) was thawed at 37° C. and washed twice with a physiological buffer containing 0.5% serum albumin. In 10 mL of DMEM/F12 containing 20% human serum (available from Gibco), 6.0×10⁷ washed cells were suspended, and then, seeded in a cell culture dish having a diameter of 10 cm (UpCell (registered trademark) 10 cm dish, CS3005, available from CellSeed Inc.). After the seeding, the cells were cultured for 20 hours in an incubator (BNA-121D, available from ESPEC Corp.) set at 37° C. and 5% CO₂. After the culturing, the culture dish was taken out from the incubator. The medium was discarded after adhesion of a sheet-shaped cell culture to the entire bottom face of the culture dish was observed. Then, the sheet-shaped cell culture was isolated from the culture dish by temperature treatment (allowed to stand at room temperature (20 to 25° C.) for 5 to 30 minutes) and pipetting.

### Comparative Example 2

### Measurement of Number of Cells in Sheet-Shaped Cell Culture Containing Skeletal Myoblast Cells

The sheet-shaped cell culture containing the skeletal myoblast cells produced in Comparative Example 1 was preserved in Hank's balanced salt solution (HBSS (+)) and DMEM at 4° C. for 72 hours, followed by measuring the number of cells.

The measurement result showed that the number of cells included in the sheet-shaped cell culture reduced to 50% or less. From the result, it is inferred that the sheet-shaped cell culture has a low cytokine production capacity.

### Example 1

### Production of Sheet-shaped Cell Culture Provided with Gel Layer

A sheet-shaped cell culture was obtained in a similar manner to Comparative Example 1. A culture solution in a culture dish was removed, and 500 μL of liquid fibrinogen (obtained by dissolving contents of vial 1 containing Bolheal® for tissue adhesion (available from Teijin Pharma Limited) (fibrinogen lyophilized powder) with contents of vial 2 (fibrinogen solution), having a fibrinogen concentration of 80 mg/mL, (the same applies hereinafter) was dripped on the sheet-shaped cell culture using a two-liquid mixing set (supplied with an applying nozzle having a length of about 6 cm and an inner diameter of about 1 mm, available from NIPRO) of a preparation set attached to Bolheal® for tissue adhesion. Then, 800 μL of liquid thrombin (obtained by dissolving contents of vial 3 containing Bolheal® for tissue adhesion (available from Teijin Pharma Limited) (thrombin lyophilized powder) with contents of vial 4 (thrombin solution), having a thrombin concentration of 250 units/mL, (the same applies hereinafter) was sprayed with a Bolheal® spray set (available from Akita Sumitomo Bakelite Co., Ltd.) at a pressure of 0.03 MPa, keeping its spray nozzle about 7 cm away from the cell sheet.

A fibrin gel is formed by a reaction between liquid fibrinogen and liquid thrombin. After standing for about 5 minutes, 24 mL of Hank's balanced salt solution (HBSS (+), Cat No. 14025, available from Life Technologies, the same applies hereinafter) was added to the culture dish and immediately removed so as to wash the culture dish containing the sheet-shaped cell culture. This process enables removal of unreacted liquid fibrinogen and liquid thrombin. Then, 24 mL of Hank's balanced salt solution was added again to the culture dish, and the sheet-shaped cell culture was allowed to stand for about 15 minutes. After that, the solution in the culture dish was removed, and the fibrin gel coagulated outside the sheet-shaped cell culture was trimmed with a scalpel, thereby isolating the sheet-shaped cell culture having a fibrin gel layer.

### Example 2

### Production of Sheet-Shaped Cell Culture Having Intercellular Gap Filled with Gel

Skeletal myoblast cells cryopreserved in a preservation solution (MCDB containing 10% DMSO) were thawed at 37° C., diluted with 10% FBS/DMEM, and centrifuged. After removing supernatant, the cells were suspended in 15 mL of DMEM containing 20% human serum at a density of 2×10⁶ cells/cm² and seeded on a temperature-responsive substrate (UpCell (registered trademark), 6 cm dish, CS3006, available from CellSeed Inc.) having a diameter of 6 cm. After the seeding, a cell population was cultured for 72 hours in an incubator (BNA-121D, available from ESPEC Corp.) set at 37° C. and 5% CO₂. After the culturing, the substrate was taken out from the incubator. The medium was discarded after adhesion of the cells to the substrate was observed.

After the medium was discarded, 500 μL of liquid fibrinogen (obtained by dissolving contents of vial 1 containing Beriplast (registered trademark) for tissue adhesion (available from CSL Behring) (fibrinogen lyophilized powder) with contents of vial 2 (fibrinogen solution, having a fibrinogen concentration of 80 mg/mL) and 500 μL of liquid thrombin (obtained by dissolving contents of vial 3 containing Beriplast (registered trademark) for tissue adhesion (available from CSL Behring K.K. Corporation) (thrombin lyophilized powder) with the contents of vial 4 (thrombin solution), having a thrombin concentration of 300 units/mL) were dripped on the substrate and allowed to stand for about 5 minutes to form a fibrin gel.

After forming the fibrin gel, 10 mL of Hank's balanced salt solution (HBSS (+), Cat No. 14025, available from Life Technologies) was added to wash the substrate three times, thereby removing unreacted fibrinogen and thrombin. Then, the substrate was allowed to stand at room temperature (20 to 25° C.) for 5 to 30 minutes to perform temperature treatment on the temperature-responsive material, followed by detaching a sheet-shaped cell culture from the substrate by pipetting, thereby isolating the sheet-shaped cell culture having an intercellular gap filled with the gel.

### Example 3

### Measurement of Number of Cells in Sheet-Shaped Cell Culture

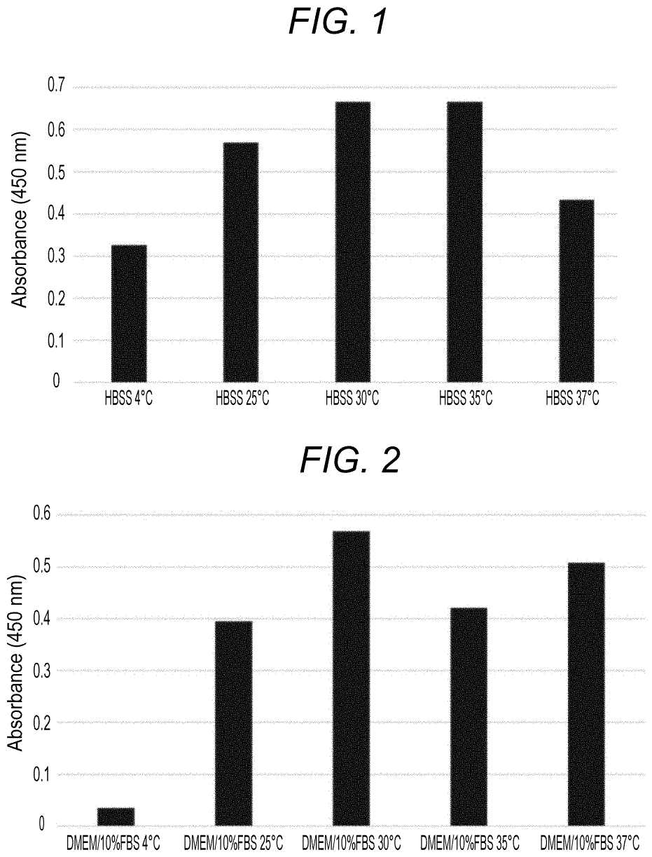

Immediately after the isolation (day 0), the cells included in the sheet-shaped cell cultures produced in Examples 1 and 2 were incubated (preserved) in Hank's balanced salt solution and in DMEM at 4° C., 25° C., 30° C., 35° C., and 37° C. for 72 hours, and the number of the cells was measured by reading the absorbance at 450 nm. FIG. 1 shows results of the sheet-shaped cell culture prepared in Example 1 incubated in Hank's balanced salt solution, and FIG. 2 shows results of the sheet-shaped cell culture incubated in DMEM. In any of the sheet-shaped cell cultures, the number of cells was found to decrease in the incubation at 4° C., increase at 25° C. or higher, and significantly increase at 30° C. or higher. The increase of the number of cells contributes to an increase in production of cytokines such as VEGF and HGF. Accordingly, these sheet-shaped cell cultures have a high cytokine production capacity. In addition, since a sheet-shaped cell culture having a scaffold as those prepared in Examples 1 and 2 has a high strength, even when the cell culture is kept incubated at 25° C. or higher, it is possible to increase the number of cells included in the graft and to enhance the cytokine production capacity without causing twist or shrinkage.

The various characteristic features described herein can be combined in various ways, and embodiments available from such combinations, inclusive of those not specifically described in the description herein, and all fall within the scope of the present disclosure. In addition, those skilled in the art are well aware of the possibility of a multiplicity of various modifications without departing from the spirit of the present disclosure, and equivalents including such modifications are encompassed within the scope of the present disclosure. Accordingly, the embodiments described herein are merely exemplifications, and it is to be understood that these embodiments are not described with an intention to restrict the scope of the present disclosure.

What is claimed is:

1. A method for enhancing activity selected from the group consisting of cytokine production capacity, proliferation capacity, engraftment capacity, angiogenesis-inducing capacity, and tissue regeneration capacity in a graft containing a somatic cell,
the method comprising incubating the graft at 25° C. or higher.

2. The method according to claim 1, wherein the graft has a scaffold.

3. The method according to claim 2, wherein the scaffold is a gel including fibrin, gelatin, or collagen.

4. A method for production of a graft, the method comprising the method according to claim 1.

5. The method according to claim 1, wherein the graft is a sheet-shaped cell culture.

6. A graft obtained by the method according to claim 1.

7. A graft obtained by the method according to claim 4.

8. A method for treating heart disease comprising using the graft according to claim 6.

9. A method for treating heart disease comprising using the graft according to claim 7.

10. A method for treating a disease to be ameliorated by application of a graft, the method comprising applying the graft according to claim 6 to a subject in need of treating the disease.

11. A method for treating a disease to be ameliorated by application of a graft, the method comprising applying the graft according to claim 7 to a subject in need of treating the disease.

12. A method for producing a graft, the method comprising:
seeding cells on a substrate;
producing a sheet-shaped cell culture using the seeded cells, the sheet-shaped cell culture forming a graft;
applying a gel layer onto the graft; and
incubating the graft at a temperature that is 25° C. or higher.

13. The method of claim 12, wherein the seeding of the cells on the substrate comprises seeding somatic cells onto the substrate.

14. The method of claim 12, wherein the applying of the gel layer onto the graft comprises either: i) simultaneously adding liquid fibrinogen and liquid thrombin onto the graft; or ii) dripping liquid fibrinogen onto the graft followed by spraying liquid thrombin onto the graft.

15. The method of claim 12, wherein the incubating of the graft at a temperature that is 25° C. or higher comprises incubating the graft at the temperature of 25° C. or higher for 1 to 96 hours.

16. The method of claim 12, wherein the seeding of the cells on the substrate comprises seeding the cells onto a substrate formed from a material selected from polyethylene, polypropylene, polyethylene terephthalate, polymethyl methacrylate, nylon 6,6, polyvinyl alcohol, cellulose, silicon, polystyrene, glass, polyacrylamide, polydimethylacrylamide, or any combination thereof.

17. The method of claim 12, wherein the incubating of the graft at a temperature that is 25° C. or higher comprises incubating the graft at a temperature from 25° C. to about 45° C.

18. The method of claim 12, wherein the applying of the gel layer onto the graft comprises adding gel to the graft until the graft possesses a thickness from 10 μm to 2000 μm.
